(19) United States
(12) Patent Application Publication
(10) Pub. No.: US 2022/0096666 A1
(43) Pub. Date: Mar. 31, 2022

(54) ULTRASOUND CONTRAST AGENT AND METHODS FOR USE THEREOF

(71) Applicant: GE Healthcare AS, Nydalen (NO)

(72) Inventors: Ingrid Henriksen, Oslo (NO); Eva Krog Tamnes, Oslo (NO); Per Sontum, Oslo (NO); Svein Kvale, Oslo (NO)

(21) Appl. No.: 17/415,842

(22) PCT Filed: Dec. 20, 2019
(86) PCT No.: PCT/EP2019/086716

§ 371 Date: Jun. 18, 2021

(30) Foreign Application Priority Data
Dec. 21, 2018 (GB) 1821049.2

(51) Int. Cl. A61K 49/22; A61K 47/02; A61K 47/24

(52) U.S. Cl. CPC A61K 49/223; A61K 47/24; A61K 47/02

(57) ABSTRACT

The present disclosure is directed to an ultrasound contrast agent comprising microbubbles of perfluorocarbon, which microbubbles are stabilised by a membrane of phospholipid; and a buffering agent; wherein the ultrasound contrast agent has a bulk pH of from about 7.5 or above, preferably about 8.5 or above. The ultrasound contrast agent is for long-term storage and is ready for use in vivo. Further disclosed is a method for preparing such an ultrasound contrast agent and methods for use of an ultrasound contrast agent in a clinical setting.

### FIELD OF THE DISCLOSURE

The present disclosure relates to the field of in vivo imaging and diagnosing of a subject, and in particular is directed to an ultrasound contrast agent which is ready for use in vivo and which can withstand long-term storage before use in vivo. The present disclosure further relates to a method for preparing an ultrasound contrast agent and methods for use of an ultrasound contrast agent in a clinical setting.

### BACKGROUND OF THE DISCLOSURE

Ultrasound contrast agents based on phospholipid-stabilised microbubbles of perfluorocarbons are well known in the art (see e.g. Wheatley et al, J. Drug Del. Sci. Technol., 23(1), 57-72, 2013). A single microbubble consists of a gas core which may be about 2-10 μm in size, encapsulated in a shell or membrane of a layer of stabilising phospholipid molecules. The gaseous core, being compressible, can expand and contract when subjected to ultrasound. The expansion and contraction of microbubbles upon exposure to ultrasound produce acoustic backscatter which is used for diagnostic imaging purposes. The microbubble surface can further be functionalised with a targeted drug moiety which is released when microbubble fractures and/or cavitation occurs upon application of ultrasound and such ultrasound contrast agents can thereby be used also in therapeutic applications (Upadhyay et al, RSC Adv., 6, 15016-15026, 2016).

Sonazoid™ is an example of an ultrasound contrast agent based on phospholipid-stabilised microbubbles of perfluorocarbons. More particularly, Sonazoid™ is formulated as a powder consisting of lyophilised sucrose entrapping microbubbles of perfluorobutane stabilised by a membrane of hydrogenated egg phosphatidyl serine, which is stored under a headspace of perfluorobutane. Sonazoid™ is aseptically produced by continuous homogenization of perfluorobutane (PFB) in an aqueous dispersion of hydrogenated egg phosphatidyl serine (HEPS). After the initial microbubble generation, the concentration and size distribution of microbubbles is adjusted through a series of controlled separation steps. The final dispersion, targeted to yield 8 μl microbubbles per ml in the reconstituted product, is made isotonic by addition of sucrose. Two ml of the dispersion is filled into 10 ml glass vials and lyophilised. After lyophilisation, the vial head space is back-filled with perfluorobutane before stoppering. In other words, Sonazoid™ is a freeze-dried product, and it has to be reconstituted with water before use. More particularly, prior to administration to a subject, the product is reconstituted by addition of 2 ml of sterile water for injection through a supplied vented filter (5 μm) spike (Codan Chemoprotect® Spike, Codan GmbH & Co., Germany) followed by manual mixing for 1 min. After reconstitution, the product appears as a milky white, homogeneous dispersion. As the dispersion is nontransparent, visual inspection for extraneous particles is difficult. To ensure the absence of such particles, the product is withdrawn through the filter spike into the syringe before administration. After reconstitution, if left non-agitated the microspheres will start to segregate by flotation and form a cream layer on top of the liquid phase. If not used immediately after reconstitution, the product should be re-homogenised prior use by manual mixing for 10 s (Sontum, Ultrasound Med. & Biol., 34(5), 824-833, 2008).

Since the active principle of an ultrasound contrast agent is a physical state (a microbubble) rather than a chemical substance, two types of stability must be considered: The physical and the chemical stability. In other words, the focus must be on how to control and maintain the microbubble concentration and size distribution, as well as the chemical composition of the components. Microbubbles are in general a thermodynamically unstable system, which may undergo physical changes during preparation and storage (see e.g. WO2015150354 A1; Segers et al, Langmuir, 33, 10329-10339, 2017; Borden et al, Advances in Colloid and Interface Science 262, 39-49, 2018). In addition, the membrane of phospholipids which stabilises the microbubbles may undergo hydrolytic degradation in solution, giving rise to impurities in the final product. Phospholipids easily undergo hydrolytic splitting in acidic and alkaline media. Only at pH 7 are phospholipids stable enough, as under these conditions the ester bond hydrolysis does not proceed to any significant degree (Phospholipids Handbook, 1993, edited by Gregor Cevc; see Chapter 9 “Chemical stability”, Evstigneeva, pp. 323-324). Temperature and pH strongly influence hydrolysis kinetics (Phospholipids Handbook, 1993, edited by Gregor Cevc; see Chapter 9 “Chemical stability”, Crommelin et al, pp. 338-339). Further, it is known that once hydrolytic degradation at low pH has been initiated, the pH drops and results in accelerated degradation. Consequently, a major challenge during early development of Sonazoid™ was how to obtain a product having an acceptable shelf life. Lyophilisation, resulting in a Sonazoid™ formulation in the form of a freeze-dried powder, has been regarded up until now as the only way of obtaining such a product.

Although the lyophilisation provides a product with excellent shelf life stability and quality, it also increases manufacturing cost significantly as it is time and resource intensive, and the “ease of use” for the end user (typically a health care professional) is reduced. Thus, there is a need in the art for improved ultrasound contrast agents based on phospholipid-stabilised microbubbles of perfluorocarbons, which ultrasound contrast agents both have high storage stability and are easy to use.

### SUMMARY OF THE DISCLOSURE

The above objective to provide ultrasound contrast agents which both have high storage stability and are easy to use is achieved by the present disclosure, which relates to a stable and ready-to-use ultrasound contrast agent formulation, in the form of a dispersion which can surprisingly withstand long-term storage before use and which is ready to use, i.e. which is ready for injection into a subject.

More particularly, the present disclosure is directed to an ultrasound contrast agent comprising:

(a) microbubbles of perfluorocarbon, which microbubbles are stabilised by a membrane of phospholipid; and

(b) a buffering agent;

wherein the ultrasound contrast agent has a bulk pH of from about 7.5 or above, preferably about 8.5 or above.

The present disclosure is also directed to a method for preparing an ultrasound contrast agent, comprising the following steps:

(i) Homogenising perfluorocarbon continuously in a sterile aqueous dispersion of phospholipid to generate phospholipid-stabilised microbubbles of perfluorocarbon dispersed in an aqueous dispersion;

(ii) Adjusting the size distribution of microbubbles in the aqueous dispersion to a median size in the range of from 1 to 6 μm, preferably from 2 to 5;

(iii) Optionally, adding a tonicity agent to the aqueous dispersion;

(iv) Adding a buffering agent to the aqueous dispersion to adjust the bulk pH of the aqueous dispersion to a pH of from about 7.5 or above, preferably about 8.5 or above;

(v) Adjusting the concentration of microbubbles in the aqueous dispersion to achieve a target concentration of microbubbles of about 6-10 μl/ml;

(vi) Dispensing the aqueous dispersion into a vial and flushing the headspace of the vial with perfluorocarbon.

Further, the present disclosure is directed to a method for improving the contrast of an ultrasonic image of tissue in a subject, a method for in vivo imaging of tissue in a subject, and a method for diagnosing of a subject, which methods comprise injecting an ultrasound contrast agent as described above into the subject.

The present disclosure also relates to an ultrasound contrast agent for use in a method as described herein.

Further, the present disclosure relates to use of an ultrasound contrast agent as disclosed herein for the manufacture of a medicament for use in a method as disclosed herein.

Preferred aspects of the present disclosure are described below in the detailed description and in the dependent claims.

### BRIEF DESCRIPTION OF THE FIGURES

FIG. 1 illustrates the chemical stability of Sonazoid bulk product taken from before freeze-drying, prepared as a non-buffered aqueous dispersion, and stored at 5° C. for 8 months.

FIG. 2 illustrates the chemical stability of freeze-dried powder of Sonazoid, prepared as a non-buffered aqueous dispersion, as a buffered aqueous dispersion comprising a buffering agent and having a bulk pH 7 at room temperature, and as a buffered aqueous dispersion comprising a buffering agent and having a bulk pH 8 at room temperature, respectively, and stored at 5° C. for 6 months.

FIG. 3 illustrates the physical stability of freeze-dried powder of Sonazoid, prepared as a non-buffered aqueous dispersion, as a buffered aqueous dispersion comprising a buffering agent and having a bulk pH 7 at room temperature, and as a buffered aqueous dispersion comprising a buffering agent and having a bulk pH 8 at room temperature, respectively, and stored at 5° C. for 6 months.

### DETAILED DESCRIPTION OF THE DISCLOSURE

The present disclosure provides a liquid ultrasound contrast agent which is storage-stable and ready-to-use. By increasing the pH and including a buffering agent in the product, a liquid formulation has been achieved, which is easier to handle for the end user compared to previously known freeze-dried powder of Sonazoid™, since no reconstitution of the powder is required before use of the presently claimed product. Here, we wish to point out that it is not evident to a person skilled in the art that adding a buffering agent to an ultrasound contrast agent based on phospholipid-stabilised microbubbles of perfluorocarbons would result in a functional ultrasound contrast agent, since electrolytes (e.g. present in a buffering agent) may change the constitution of the dispersion. Nevertheless, the present inventors have surprisingly managed to keep the volume concentration and distribution of microbubbles at a desired level, i.e. have managed to maintain the physical stability while improving the chemical stability of the product, compared to the physical stability and chemical stability, respectively, of a previously known dispersion of reconstituted freeze-dried powder of Sonazoid™. Consequently, the storage stability of the presently claimed product is improved compared to the previously known product.

By the inclusion of a buffering agent in the formulation, the ultrasound contrast agent according to the present disclosure has a bulk pH which lies in the alkaline range at a temperature of 5° C. As shown in the examples further below, the alkaline pH significantly decreases the rate of hydrolysis of the phospholipids present in the ultrasound contrast agent, which thus stays chemically stable during a much longer period. The chemical stability of the phospholipids further influences the physical stability of the microbubbles since the membrane of phospholipids stabilises the microbubbles.

More particularly, the present disclosure solves or at least mitigates the problems associated with existing ultrasound contrast agents based on phospholipid-stabilised microbubbles of perfluorocarbons by providing an ultrasound contrast agent comprising:

(a) microbubbles of perfluorocarbon, which microbubbles are stabilised by a membrane of phospholipid; and

(b) a buffering agent;

wherein the ultrasound contrast agent has a bulk pH of from about 7.5 or above, preferably about 8.5 or above.

The term “contrast agent” has its conventional meaning in the field of in vivo medical imaging, and refers to an agent in a form suitable for mammalian administration, which assists in providing clearer images in the region or organ of interest than could be obtained by imaging the mammalian subject alone. By the term “subject” is meant a mammal in vivo, preferably the intact mammalian body in vivo, and more preferably a living human subject. By the phrase “in a form suitable for mammalian administration” is meant a composition which is sterile, pyrogen-free, lacks compounds which produce toxic or adverse effects, and is formulated at a biocompatible pH (approximately pH 4.0 to 10.5). Such compositions lack particulates which could risk causing emboli in vivo, and are formulated so that precipitation does not occur on contact with biological fluids (e.g. blood). Such compositions also contain only biologically compatible excipients, and are preferably isotonic.

As with other in vivo imaging agents, the contrast agent is designed to have minimal pharmacological effect on the mammalian subject to be imaged. Preferably, the contrast agent can be administered to the mammalian body in a minimally invasive manner, i.e. without a substantial health risk to the mammalian subject when carried out under professional medical expertise. Such minimally invasive administration is preferably intravenous administration into a peripheral vein of said subject, without the need for local or general anaesthetic.

The term “microbubble” has its conventional meaning in the field of in vivo ultrasound imaging, and refers to a gas microbubble having an inner diameter of 0.1-10 μm, typically between 0.5 and 5 μm. Such microbubbles are similar in size to a red blood cell, which allow them to display similar characteristics in the microvessels and capillaries throughout the mammalian body (Sirsi et al, Bubble Sci. Eng. Technol, 1(1-2), 3-17, 2009). Herein, the terms “microbubble” and “microsphere” may be used interchangeably.

The term “perfluorocarbons” has its conventional chemical meaning, and is a generic term for a group of organofluorine compounds with the formula CₓF_{y}, i.e. they contain only carbon and fluorine (see IUPAC, Compendium of Chemical Terminology, 2nd ed., 1997; online corrected version, 2006-). Compounds with the prefix perfluoro- are hydrocarbons, including those with heteroatoms, wherein all C—H bonds have been replaced by C—F bonds. Perfluorocarbons include perfluoroalkanes, fluoroalkenes, fluoroalkynes and perfluoroaromatic compounds. The terms “perfluorocarbon” and “fluorocarbon” may be used interchangeably. Suitable perfluorocarbons according to the present disclosure include perfluoroalkanes, e.g. perfluorobutane, perfluoropropane, and perfluoropentane. Presently preferred perfluorocarbon of the present disclosure is perfluorobutane (“PFB”), which has its standard chemical meaning, and is also referred to in the context of medical uses as perflubutane. The chemical formula of perfluoro-n-butane is CF₃CF₂CF₂CF₃ or C₄F₁₀, with a boiling point of −2.2° C. Commercial perfluoro-n-butane contains a minor amount (typically 2-4%) of the perfluoro-iso-butane isomer, i.e. C₄HF₉.

Suitable microbubbles according to the present disclosure include microbubbles of perfluorocarbon which are stabilised by a membrane of phospholipid, as described e.g. in Sontum (above) and Sirsi et al (above). A suitable membrane (or shell or coating) of phospholipid according to the present disclosure has a net negative charge. Presently preferred phospholipids are the phospholipids present in hydrogenated egg phosphatidylserine (HEPS), i.e. primarily phosphatidylserine and phosphatidic acid (Hvattum et al, J. Pharm. Biomed. Anal., 42(4), 506-512, 2006). The membrane of phospholipid typically has a thickness of 10 to 100 nm.

Herein, the term “buffering agent” refers to a buffer, which is a solution containing either a weak acid and its salt or a weak base and its salt, and which is resistant to changes in pH. In other words, a buffer is an aqueous solution of either a weak acid and its conjugate base or a weak base and its conjugate acid. Buffering agents are used to maintain a stable pH in a solution (or suspension or dispersion), as they can neutralize small quantities of additional acid or base. The buffering agent is chosen from any buffering agent, which is physiologically compatible and suitable for injection in vivo into a subject. Examples of suitable buffering agents according to the present disclosure are tris(hydroxymethyl) aminomethane (abbrev. Tris), sodium phosphate, ammonium chloride, diethanolamine, glycine, triethanolamine, and sodium carbonate.

A presently preferred buffering agent is Tris. The pH of Tris is temperature-dependent. At low temperatures, the pH of Tris is higher than at higher temperatures. For example, if a Tris buffer has a pH of 8.26 at 5° C., the Tris buffer will have a pH of 7.7 at 25° C. and a pH of 7.4 at 37° C. Storage of an ultrasound contrast agent is preferably done in a refrigerated space, i.e. at a temperature of approx. 3-6° C., which helps maintain the physical and chemical stability of the ultrasound contrast agent. As explained and shown elsewhere herein an alkaline pH also helps maintain the chemical stability and physical stability of the ultrasound contrast agent. At the same time, an ultrasound contrast agent should preferably have a pH close to the physiological pH of 7.4 when injected in vivo into a subject. The fact that the pH of Tris is temperature-dependent can thus be used as an advantage of an ultrasound contrast agent according to the present disclosure since refrigerated storage will be at a higher pH than the pH at body temperature.

The term “bulk pH” refers to the pH of a solution (or suspension or dispersion) as measured inside the bulk or volume of the solution, such as at or close to the center of the volume of the solution, as opposed to at a surface of the solution. The bulk pH can differ from the surface pH of a solution. An ultrasound contrast agent according to the present disclosure has a bulk pH in the alkaline range at a temperature of 5° C., i.e. has a bulk pH of about 7.5 or above at a temperature of 5° C., suitably at most 10.0 at a temperature of 5° C., such as about 7.5, 7.75, 8.0, 8.25, 8.5, 8.75, 9.0, 9.25, 9.5, 9.75, or 10.0, at a temperature of 5° C. Presently preferred bulk pH is about 8.25-9.25 at a temperature of 5° C., such as 8.25, 8.5, 8.75, 9.0, or 9.25 at a temperature of 5° C. In this context, throughout the text, the term “about” is intended to mean that all pH values mentioned herein may generally vary about 0.1-0.5, i.e. ±0.1-0.5, such as ±0.1, ±0.2, ±0.3, ±0.4, or ±0.5.

The ultrasound contrast agent of the invention is preferably stored at a low temperature, particularly for longer storage periods. For storage periods up to about 1 month temperatures of up to room temperature can be suitable. It is preferred that the storage temperature is no lower than the freezing point of the ultrasound contrast agent, and more preferred that it is above said freezing point as a solution. An exemplary temperature range for storage of the ultrasound contrast agent of the invention would be above its freezing point and up to around 5° C. When in use the ultrasound contrast agent of the invention will be brought to ambient temperature prior to administration to a subject.

The ultrasound contrast agent according to the present disclosure is for long-term storage, i.e. can withstand long-term storage. In other words, the ultrasound contrast agent maintains its physical stability and chemical stability during long-term storage, i.e. has an acceptable or even excellent shelf life. In this context, throughout the text, “long-term” is intended to mean a period of months or years, such as a period of 1, 2, 3, 4, 5, 6, 7, 8, 9, 10, 11, 12, 13, 14, 15, 16, 17, 18, 19, 20, 21, 22, 23, or 24 months. Long-term storage preferably occurs at a temperature of approx. 3-6° C., such as at 3, 4, 5, or 6° C.

The ultrasound contrast agent according to the present disclosure differs from the current commercially-available freeze-dried formulations as it is ready to use directly from the vial as purchased, where “ready to use” means ready to use in a clinical setting, such as ready to use for injection into a patient for in vivo imaging, diagnosing and/or treatment of a subject.

The ultrasound contrast agent according to the present disclosure is in liquid form, i.e. is a liquid formulation, particularly in the form of a dispersion, such as an aqueous dispersion, as defined elsewhere herein. The liquid formulation is for long-term storage and is ready to use in a clinical setting.

The buffering agent included in the ultrasound contrast agent according to the present disclosure may be selected from a group consisting of tris(hydroxymethyl)aminomethane (Tris), sodium phosphate, ammonium chloride, diethanolamine, glycine, triethanolamine, and sodium carbonate.

Further, the buffering agent may have a concentration of from about 1 mM to about 10 mM, such as 1, 2, 3, 4, 5, 6, 7, 8, 9, or 10 mM. In this context, throughout the text, the term “about” is intended to mean that all concentration values mentioned herein may generally vary about 0.1-0.5 mM, i.e. ±0.1-0.5, such as ±0.1, ±0.2, ±0.3, ±0.4, or ±0.5 mM.

The membrane of phospholipid included in the ultrasound contrast agent according to the present disclosure preferably has a net negative charge.

A presently preferred ultrasound contrast agent according to the present disclosure comprises microbubbles of perfluorobutane stabilised by hydrogenated egg phosphatidyl serine, like Sonazoid™ (GE Healthcare AS), previously known as NCI 00100, as described by Sontum (above), and further comprises tris(hydroxymethyl)aminomethane (i.e. Tris) as the buffering agent.

The ultrasound contrast agent according to the present disclosure may further comprise a tonicity agent, i.e. an excipient added to make the ultrasound contrast agent isotonic. Examples of tonicity agents are salts of plasma cations with biocompatible counterions, sucrose, saline, dextrose, glycerin and mannitol.

The ultrasound contrast agent according to the present disclosure may, alternatively or additionally, comprise a viscosity agent, i.e. an excipient added to change the viscosity of the ultrasound contrast agent, and/or a flotation-reducing agent, e.g. propylene glycol, glycerol, glycerin, and/or polyethylene glycol.

The present disclosure is also directed to a method for preparing an ultrasound contrast agent, comprising the following steps:

(i) Homogenising perfluorocarbon continuously in a sterile aqueous dispersion of phospholipid to generate phospholipid-stabilised microbubbles of perfluorocarbon dispersed in an aqueous dispersion;

(ii) Adjusting the size distribution of microbubbles in the aqueous dispersion to a median size in the range of from 1 to 6 μm, preferably from 2 to 5 μm;

(iii) Optionally adding a tonicity agent to the aqueous dispersion;

(iv) Adding a buffering agent to the aqueous dispersion to adjust the bulk pH of the aqueous dispersion to a pH of from about 7.5 or above, preferably about 8.5 or above;

(v) Adjusting the concentration of microbubbles in the aqueous dispersion to achieve a target concentration of microbubbles of about 6-10 μl/ml, such as about 6, 7, 8, 9, or 10 μl/ml, presently preferably about 8 μl/ml;

(vi) Dispensing the aqueous dispersion into a vial and flushing the headspace of the vial with perfluorocarbon.

In other words, the present disclosure is directed to a method for preparing an ultrasound contrast agent, comprising the following steps:

(i) Homogenising perfluorocarbon continuously in a sterile aqueous dispersion of phospholipid to generate phospholipid-stabilised microbubbles of perfluorocarbon dispersed in an aqueous dispersion;

(ii) Adjusting the size distribution of microbubbles in the aqueous dispersion to a median size in the range of from 1 to 6 μm, preferably from 2 to 5 μm;

(iii) Optionally adding a tonicity agent to the aqueous dispersion;

(iv) Adding a buffering agent to the aqueous dispersion to adjust the bulk pH of the aqueous dispersion to a pH of from about 7.5 or above, preferably about 8.5 or above;

(v) Adjusting the concentration of microbubbles in the aqueous dispersion to achieve a target concentration of microbubbles of about 6-10 μl/ml, such as about 6, 7, 8, 9, or 10 μl/ml, presently preferably about 8 μl/ml;

(vi) Dispensing the aqueous dispersion into a vial and flushing the headspace of the vial with perfluorocarbon;

with the proviso that no lyophilisation of the dispersion is performed or required before long-term storage and/or injection in vivo of the ultrasound contrast agent.

The term “dispersion”, as in “aqueous dispersion”, is intended to mean a composition in which one substance is dispersed within another substance. How dispersions are classified can vary, with the two main approaches to classification being (1) the nature of the dispersion's internal and external phases (e.g., solid, liquid, or gas) and (2) the size range of its dispersed particles (colloidal versus coarse).

The term “suspension” has been used to describe the previously known Sonazoid formulation (see e.g. WO2015150354A1). However, since the term “suspension” when used pharmaceutically is now generally used mainly for solid particles dispersed in an external phase, herein the term “dispersion” is preferably used for the newly disclosed liquid formulation comprising gas dispersed in an external phase. Nevertheless, the terms “dispersion” and “suspension” may be used interchangeably herein.

The term “aqueous dispersion” refers to a dispersion of the microbubbles in an aqueous solvent, which comprises water and/or water-miscible solvents. The aqueous solvent is preferably a biocompatible carrier. By the term “biocompatible carrier” is meant a fluid, especially a liquid, such that the composition is physiologically tolerable, i.e. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is isotonic). One or more excipients may be added to the biocompatible carrier as is well-known to those of skill in the art, such as: an aqueous buffer solution comprising a biocompatible buffering agent (e.g. phosphate buffer); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). Preferably the biocompatible carrier is pyrogen-free water for injection or isotonic saline. Hence the aqueous dispersion suitably excludes water-immiscible organic solvents.

The phrase “adjust the bulk pH of the aqueous dispersion to a pH of from about 7.5 or above” is intended to mean that the bulk pH of the aqueous dispersion is adjusted to a pH of from about 7.5 or above preferably as measured at a particular temperature, e.g. 5° C.

Herein, “target concentration” is defined as the concentration after long-term storage and/or the concentration at the time of injection in vivo in a subject. Upon preparation of the ultrasound contrast agent, the concentration of microbubbles may decrease initially and stabilize at a slightly lower concentration. The target concentration is obtained based on appropriate dilution of a known size distribution of stabilised microbubbles.

The target concentration of microbubbles is about 6-10 μl/ml, preferably about 8 μl/ml. In this context, throughout the text, the term “about” is intended to mean that the concentration values mentioned herein may generally vary about 0.1-0.5 μl/ml, i.e. ±0.1-0.5 μl/ml, such as ±0.1, ±0.2, ±0.3, ±0.4, or ±0.5 μl/ml.

When dispensing the aqueous dispersion into a vial according to step (vi) described above, the vial is normally not filled to the top but only partly, thereby leaving a headspace above the dispersion which can be flushed (i.e. filled) with a headspace gas. The term “headspace” has its conventional meaning and refers to the gas within the vial above the aqueous dispersion. Suitable types of vials or containers in which the aqueous dispersion may be stored include injection vials (e.g. plastic or glass, opaque or clear), such as vials with surface coating (e.g. to prevent ionic leachables). Also contemplated are ready-made syringes prefilled with the ultrasound contrast agent, which would obviate the need for withdrawing the ultrasound contrast agent from a vial before injecting it into a subject.

In the above-described method for preparing an ultrasound contrast agent, steps (iii) and (iv) may be performed in any order.

In the above-described method for preparing an ultrasound contrast agent, step (v) may be performed before or after any one of steps (ii), (iii) and (iv), with the proviso that step (v) is performed after step (i) and before step (vi).

The ultrasound contrast agent prepared according to the above-described method is for long-term storage and/or is ready to use in a clinical setting, i.e. is ready to use in vivo, such as for in vivo imaging, diagnosing and/or treatment of a subject.

The present disclosure is further directed to a method for improving the contrast of an ultrasonic image of tissue in a subject, comprising injecting the ultrasound contrast agent according to any one of the aspects and embodiments described above into said subject and carrying out an ultrasound scan of said tissue.

The present disclosure is also directed to a method for in vivo imaging of tissue in a subject, comprising injecting the ultrasound contrast agent according to any one of the aspects and embodiments described above into said subject, carrying out an ultrasound scan of said tissue and generating an image of said tissue.

Further, the present disclosure is directed to a method for diagnosing of a subject, such as in vivo diagnosing of a subject, comprising injecting the ultrasound contrast agent according to any one of the aspects and embodiments described above into said subject, carrying out an ultrasound scan of a region of interest in said subject, generating an image of said region of interest and assessing said image in order to make a diagnosis.

The present disclosure is also directed to an ultrasound contrast agent for use in a method for improving the contrast of an ultrasonic image of tissue in a subject, comprising injecting the ultrasound contrast agent according to any one of the aspects and embodiments described above into said subject and carrying out an ultrasound scan of said tissue.

Further, the present disclosure is directed to an ultrasound contrast agent for use in a method for in vivo imaging of tissue in a subject, comprising injecting the ultrasound contrast agent according to any one of the aspects and embodiments described above into said subject, carrying out an ultrasound scan of said tissue and generating an image of said tissue.

The present disclosure also relates to an ultrasound contrast agent for use in a method for in vivo diagnosing of a subject, comprising injecting the ultrasound contrast agent according to any one of the aspects and embodiments described above into said subject, carrying out an ultrasound scan of a region of interest in said subject, generating an image of said region of interest and assessing said image in order to make a diagnosis.

The present disclosure further is directed to use of an ultrasound contrast agent according to any one of the aspects and embodiments described above for the manufacture of a medicament for improving the contrast of an ultrasonic image of tissue in a subject (comprising injecting the ultrasound contrast agent according to any one of the aspects and embodiments described above into said subject and carrying out an ultrasound scan of said tissue).

Also, the present disclosure relates to use of an ultrasound contrast agent according to any one of the aspects and embodiments described above for the manufacture of a medicament for in vivo imaging of tissue in a subject (comprising injecting the ultrasound contrast agent according to any one of the aspects and embodiments described above into said subject, carrying out an ultrasound scan of said tissue and generating an image of said tissue).

Further, the present disclosure is directed to use of an ultrasound contrast agent according to any one of the aspects and embodiments described above for the manufacture of a medicament for in vivo diagnosing of a subject (comprising injecting the ultrasound contrast agent according to any one of the aspects and embodiments described above into said subject, carrying out an ultrasound scan of a region of interest in said subject, generating an image of said region of interest and assessing said image in order to make a diagnosis).

Compositions “comprising” one or more recited elements may also include other elements not specifically recited. The term “comprising” includes as a subset “consisting essentially of” which means that the composition has the components listed without other features or components being present.

The singular “a” and “an” shall be construed as including also the plural.

The main impurities found in previously known Sonazoid™ powder for injection are degradation products of the phospholipids, resulting from hydrolysis of phosphatidylserine sodium salt (PS) and phosphatidic acid sodium salt (PA), the two components of the HEPS-Na excipient. The hydrolytic degradation of the excipient HEPS-Na is mainly taking place during the autoclaving of the hydrated phospholipid suspension. The major degradation products are free fatty acids (FFA), lyso-phosphatidylserine sodium salt (lyso-PS) and lysophosphatidic acid sodium salt (lyso-PA). PA is present as a component in HEPS-Na, but may also be a degradation product of PS. Diacylglycerol (diacyl-G) is another phospholipid related degradation product. In the following examples, the main parameter used as a measure of the chemical stability of the product is the free fatty acids (FFA) as a percentage of the phosphatidylserine (PS) and phosphatidic acid (PA) present at each time of measurement. Also the presence of lyso-PS and lyso-PA has been measured but the corresponding data is only shown in relation to one of the examples. The main parameters used in the following examples as a measure of the physical stability of the product are the volume concentration and median size of the microbubbles.

### Example 1

Leftover from the filling line prior to lyophilisation from commercial Sonazoid production were used to generate samples. Bulk product was temporally stored in 20 L Sartorius Stedim Flexboy bags and then filled in a LAF bench to four different sterile vials types, followed by flushing head space with perfluorobutane (PFB). Two different Sonazoid batches were tested (batch 1, batch 2).

As the physical stability of the microbubbles is the parameter most likely to be affected by a liquid formulation, microbubble content and microbubble size vs. storage time were evaluated. Primary responses were parameters from the assay analysis by Coulter counting; number and volume concentrations and number and volume weighted mean diameters/distributions. In addition, microbubble morphology (shape, structure, agglomeration, foreign material etc.) were evaluated by microscopy/image analysis, and a chemical analysis of lipid content and purity was performed at the sampling point at 6 months (batch 1) or 8 months (batch 2) of storage, respectively. All samples were stored at 5° C.

### Stability Results

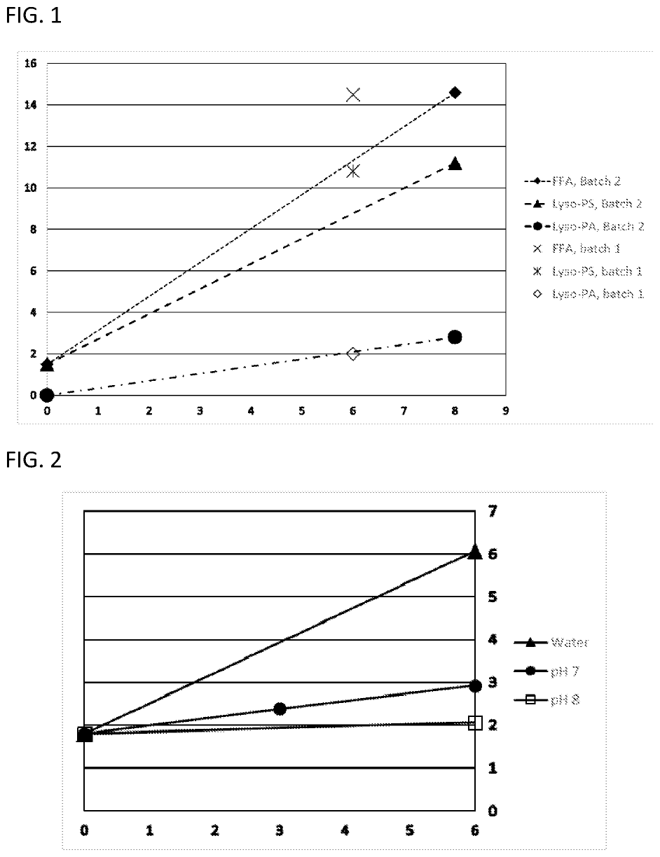

The physical stability of the microbubbles was surprisingly stable even 6 months (batch 1, batch 2) after manufacture of the Sonazoid aqueous dispersion. However, as shown in FIG. 1 the hydrolysis of the phospholipids was significant after 6 months (batch 1) and 8 months (batch 2). FIG. 1 shows the degree of degradation of phospholipids due to hydrolysis in samples of Sonazoid bulk product taken from before freeze-drying, prepared as a non-buffered aqueous dispersion and stored at 5° C. for 6-8 months (months on the x-axis). The degree of hydrolysis is illustrated by the presence of three degradation products; free fatty acids (FFA), lyso-phosphatidylserine (lyso-PS), and lyso-phosphatidic acid (lyso-PA), as a percentage of the sum of phosphatidylserine (PS) and phosphatidic acid (PA) present at each time of analysis, respectively (% FFA of (PS+PA) on the y axis).

Further, after 6 months of storage (batch 1, batch 2), the pH had decreased from approx. 6-7 to approx. 4.9-6.4, i.e. a decrease in all samples, which was expected in view of the significant hydrolysis of phosphatidylserine.

A conclusion drawn from this study is that hydrolysis must be significantly slower in order to obtain an acceptable shelf life of a ready to use formulation and the critical level of hydrolysis must likely be based on the documented effect that the hydrolytic impurities have on microbubble properties.

Example 1 above suggested that ready to use Sonazoid is not obtainable with the existing formulation (i.e. when stored in water), due to significant chemical degradation. The existing Sonazoid formulation contains no buffer, and the pH is typically about 6 to 7. After significant hydrolysis according to Example 1 above, the pH decreased to 4.9 to 6.4.

Literature data relating to liposome dispersions suggest that phospholipid hydrolysis is influenced by pH (Grit et al, Biochim. Biophys. Acta, 1167, 49-55, 1993). However, buffers that are used with liposomes are not necessarily compatible with microbubbles and stability data obtained with liposomes are not necessarily transferable to phospholipid-stabilised microbubbles, since phospholipid-stabilised microbubbles are different from liposomes in several ways. They contain a single stabilising monolayer and there is no transport of water apart from gas molecules between the external phase and the internal phase. Physically, microbubbles tend to float due to the large difference between inner and outer phases, whereas small unilamellar liposomes may be physically homogenous during storage with no apparent sedimentation. Microbubbles may therefore need additional surface stabilisation or charge in order to avoid coalescence during storage. Added ions will shield the surface charge and would be expected to reduce the physical stability of the dispersion.

Nevertheless, the present inventors decided to perform a second study of shelf life stability in which it was tested whether the degradation would be significantly slowed down by increasing the pH to neutral or basic, and by adding a buffer that prevents decrease of pH due to initial hydrolysis. The study design and stability results of said study are disclosed in Example 2 below.

### Example 2

To control and stabilise the pH, a 5 mM tris(hydroxymethyl)aminomethane (Tris) buffer was used to obtain two different test dispersions; one buffered aqueous dispersion of Sonazoid having a bulk pH of 7.5 at 5° C. (corresponding to about pH 7 at room temperature since the pH of Tris is temperature dependent, as described elsewhere herein), and one buffered aqueous dispersion of Sonazoid having a bulk pH of 8.5 at 5° C. (corresponding to about pH 8 at room temperature), respectively. Further, Sonazoid in a non-buffered aqueous dispersion was used as reference.

Freeze-dried Sonazoid was used for preparation of samples. Stock buffer solution was made from 1 M Tris solution with pH 7.0 & 8.0 (at room temperature) from Invitrogen buffer kits (Termo Fisher Scientific). The Tris buffer was diluted in a 100 ml Water For Injection (WFI) Ecoflac bottle from B. Braun.

Making up 5 mM Tris buffer 7 pH (at room temperature) Sonazoid vials:
Withdraw 0.5 ml using a syringe with sterile filter from 1 M Tris pH 7.0 buffer kit Invitrogen by Thermo Fisher Scientific AM9850G Ambion, and inject it into GE Healthcare Ecoflac WFI 100 ml produced by B. Braun Medical SA. Shake the bottle to get a homogeneous buffer solution. Reconstitute 20 Sonazoid vials, venting the vials with a sterile filter.

Making up 5 mM Tris buffer 8 pH (at room temperature) Sonazoid vials:
Withdraw 0.5 ml using a syringe with sterile filter from 1 M Tris pH 8.0 buffer kit Invitrogen by Thermo Fisher Scientific AM9850G Ambion, and inject it into GE Healthcare Ecoflac WFI 100 ml produced by B. Braun Medical SA. Shake the bottle to get a homogeneous buffer solution. Reconstitute 20 Sonazoid vials, venting the vials with a sterile filter.

For comparison, 15 vials of Sonazoid were reconstituted with water for injection from GE Healthcare Ecoflac WFI 100 ml produced by B. Braun Medical SA. All vials were stored at 5° C. after reconstitution.

### Stability Results

Responses selected were microbubble size and concentration (by Coulter Counting) and purity (by thin layer chromatography, TLC) and pH.

FIG. 2 shows the degree of degradation of phospholipids due to hydrolysis in samples of freeze-dried Sonazoid powder reconstituted with non-buffered water for injection, a buffering agent at pH 7 (at room temperature) or a buffering agent at pH 8 (at room temperature), respectively, and stored at 5° C. for 6 months (months on the x axis). The degree of hydrolysis is illustrated by the degradation product free fatty acids (FFA) as a percentage of the sum of phosphatidylserine (PS) and phosphatidic acid (PA) present at each time of analysis, respectively (% FFA of (PS+PA) on the y axis).

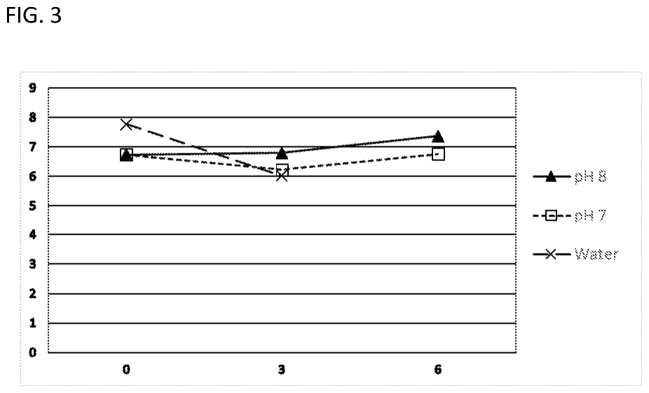

FIG. 3 shows the volume concentration of microbubbles during storage at 5° C. up to 6 months (months on the x axis; volume concentration in μl/ml on the y axis). The figure shows the average result of 10 samples for each data point. The variation between data points was normal analytical variation and no trend was visible with regards to change in volume concentration after 6 months of storage. Thus, the volume concentration was stable during 6 months of storage.

The median size of microbubbles was also found to be stable during 6 months of storage (data not shown).

The pH values of the two test dispersions at time zero, after 3 months and 6 months, respectively, are shown in Table 1 below.

TABLE 1
pH values

| Vial no. | Zero | 3 M | 6 M |
|---|---|---|---|
| pH 8 start | 8.04 | 7.97 | 8.08 |
| pH 7 start | 7.28 | 7.26 | 7.38 |

### Example 4

An ultrasound contrast agent according to the present disclosure is prepared as follows. Microbubbles are formed by homogenizing perfluorobutane in a sterile aqueous dispersion of HEPS Sodium to generate HEPS stabilized microbubbles of PFB dispersed in water. The microbubble size distribution is adjusted by repeated flotation to remove smaller microbubbles and obtain a median size of between 1 to 6 μm. The dispersion is diluted with water. Optionally, the tonicity is adjusted with a tonicity agent, such as sucrose.

The pH of the dispersion is adjusted to a desirable alkaline pH, such as about 7.5 or above, by adding Tris to 5 mM concentration.

The target concentration is obtained based on appropriate dilution of a known size distribution of stabilised microbubbles. This may for example be done as follows. The concentration of microbubbles is adjusted to achieve a target concentration of microbubbles after storage of about 6-10 μl/ml, e.g. by adjusting the concentration of microbubbles to between 8-20 μl/ml.

The dispersion is filled into a 2-10 ml vial and the headspace flushed with PFB before stoppering and capping.

The vials are stored refrigerated.

### Discussion

% FFA was used as marker for differences in hydrolysis between samples. The % FFA in the samples containing Tris buffer in Example 2 were significantly lower after 6 months of storage compared to the % FFA in the non-buffered aqueous solutions of Examples 1 and 2. All other purity parameters measured in the study of Example 2 were stable after 6 months (data not shown). The data indicate that increasing the pH and stabilizing it with a buffer during storage has a significant effect in reducing hydrolysis. At pH 8.5 at 5° C., a shelf life of 1-2 years may be possible depending on kinetics (30 months+ if the rate of hydrolysis is linear, based on the results shown in FIG. 2).

Example 1 showed that phospholipid stabilised perfluorocarbon microbubbles that are stored in water for 6 months at 5° C. undergo a significant hydrolysis of the phospholipids, which also affects the physical stability of the microbubbles with a decrease in volume concentration after 6 months. In comparison, Example 2, where microbubbles are stored for 6 months at 5° C. but at a pH above 7.5 or 8.5 in an aqueous dispersion containing a buffer, showed significantly less hydrolysis and no effect on the physical stability. Addition of small amounts of buffer gave no visible aggregation although addition of ions is known to reduce repulsion between individual microbubbles. Also, the physical microbubble parameters such as volume concentration and median size were not affected after 6 months.

The results show that the addition of a buffering agent to increase the pH of an ultrasound contrast agent in the form of a dispersion is a feasible way to decrease the rate of degradation of negatively charged phospholipids significantly. At the same time, the results show that the visual appearance or the volume concentration of the microbubbles is not affected by the addition of a buffering agent. Thus, it has been shown that both the physical stability and the chemical stability of the dispersion are maintained at a physiologically acceptable level during storage. Consequently, the present disclosure provides an ultrasound contrast agent which is both ready to use (meaning ready to inject in vivo in a subject) and which withstands long-term storage before such use.

It is to be understood that the present disclosure is not restricted to the above-described exemplifying embodiments thereof and that several conceivable modifications of the present disclosure are possible within the scope of the following claims.

1. An ultrasound contrast agent comprising:
(a) microbubbles of perfluorocarbon, which microbubbles are stabilised by a membrane of phospholipid; and
(b) a buffering agent;
wherein the ultrasound contrast agent has a bulk pH of from about 7.5 or above.

2. The ultrasound contrast agent according to claim 1, which is suitable for long-term storage.

3. The ultrasound contrast agent according to claim 1, which is suitable for use in a clinical setting.

4. The ultrasound contrast agent according to claim 1, wherein the buffering agent is selected from tris(hydroxymethyl)aminomethane (Tris), sodium phosphate, ammonium chloride, diethanolamine, glycine, triethanolamine, or sodium carbonate.

5. The ultrasound contrast agent according to claim 1, wherein the buffering agent has a concentration of from about 1 mM to about 10 mM.

6. The ultrasound contrast agent according to claim 1, wherein the membrane of phospholipid has a net negative charge.

7. The ultrasound contrast agent according to claim 1, wherein the ultrasound contrast agent further comprises a tonicity agent.

8. The ultrasound contrast agent according to claim 1, wherein the ultrasound contrast agent further comprises a viscosity agent, and/or a flotation-reducing agent.

9. The ultrasound contrast agent according to claim 1, wherein the perfluorocarbon is selected from perfluorobutane, perfluoropropane, armor perfluoropentane.

10. The ultrasound contrast agent according to claim 1, wherein the perfluorocarbon is perfluorobutane and the phospholipid is hydrogenated egg phosphatidyl serine.

11. A method for preparing an ultrasound contrast agent, comprising the following steps:
(i) Homogenising perfluorocarbon continuously in a sterile aqueous dispersion of phospholipid to generate phospholipid-stabilised microbubbles of perfluorocarbon dispersed in an aqueous dispersion;
(ii) Adjusting the size distribution of microbubbles in the aqueous dispersion to a median size in the range of from 1 to 6 μm;
(iii) Optionally adding a tonicity agent to the aqueous dispersion;
(iv) Adding a buffering agent to the aqueous dispersion to adjust the bulk pH of the aqueous dispersion to a pH of from about 7.5 or above;
(v) Adjusting the concentration of microbubbles in the aqueous dispersion to achieve a target concentration of microbubbles of about 6-10 μl/ml; and
(vi) Dispensing the aqueous dispersion into a vial and flushing the headspace of the vial with perfluorocarbon.

12. The method according to claim 11, wherein steps (iii) and (iv) are performed in any order.

13. The method according to claim 11, wherein step (v) is performed before or after any one of steps (ii), (iii) and (iv), with the proviso that step (v) is performed after step (i) and before step (vi).

14. The method according to claim 11, wherein the ultrasound contrast agent is for long-term storage and/or is ready to use in a clinical setting.

15. The method according to claim 11, which does not include a step of freeze-drying.

16. An ultrasound contrast agent prepared according to the method as defined in claim 11.

17. A method for improving the contrast of an ultrasonic image of tissue in a subject comprising injecting the ultrasound contrast agent according to claim 1 into said subject and carrying out an ultrasound scan of said tissue.

18. A method for in vivo imaging of tissue in a subject, comprising injecting the ultrasound contrast agent according to claim 1 into said subject, and carrying out an ultrasound scan of said tissue and generating an image of said tissue.

19. A method for diagnosing of a subject, comprising injecting the ultrasound contrast agent according to claim 1 into said subject, carrying out an ultrasound scan of a region of interest in said subject, and generating an image of said region of interest and assessing said image in order to make a diagnosis.

20. An ultrasound contrast agent for use in a method according to claim 11.

21. (canceled)
